(19) United States
(12) Patent Application Publication
(10) Pub. No.: US 2022/0137053 A1
(43) Pub. Date: May 5, 2022

(54) LIPID BIOMARKERS FOR CANCER SCREENING AND MONITORING

(71) Applicant: Arizona Board of Regents on Behalf of the University of Arizona, Tucson, AZ (US)

(72) Inventors: Floyd Chilton, Tucson, AZ (US); Ashley Jones Snider, Tucson, AZ (US); Justin Michael Snider, Tucson, AZ (US)

(21) Appl. No.: 17/310,274

(22) PCT Filed: Jan. 29, 2020
(86) PCT No.: PCT/US2020/015617

§ 371 Date: Jul. 27, 2021

Related U.S. Application Data
(60) Provisional application No. 62/798,606, filed on Jan. 30, 2019.

(51) Int. Cl. G01N 33/574

(52) U.S. Cl. CPC G01N 33/57434; G01N 33/57423; G01N 33/5743; G01N 2405/08; G01N 33/57446; G01N 33/57449; G01N 33/57442

(57) ABSTRACT

Provided herein are biomarkers for cancer screening and monitoring. In particular, provided herein are lipid biomarkers for cancer diagnosis, prognosis, risk, and response to treatment.

### CROSS-REFERENCE TO RELATED APPLICATION

This application claims priority to and the benefit of U.S. Provisional Application No. 62/798,606, filed Jan. 30, 2019, which is hereby incorporated by reference in its entirety.

### STATEMENT REGARDING FEDERALLY SPONSORED RESEARCH OR DEVELOPMENT

This invention was made with government support under Grant No. R01 AT008621 awarded by the National Institutes of Health. The government has certain rights in the invention.

### FIELD OF THE INVENTION

Provided herein are biomarkers for cancer screening and monitoring. In particular, provided herein are lipid biomarkers for cancer diagnosis, prognosis, risk, and response to treatment.

### BACKGROUND

Current methods used to screen for and diagnose prostate cancer (prostate cancer) severity include the Prostate Specific Antigen (PSA) test, as well as the Gleason Score, which is a grading system for prostate biopsy samples used to determine the aggressiveness of prostate cancer. There has been controversy surrounding the value of these tests, particularly the PSA test. It has been demonstrated that the harms resulting from PSA testing, such as unnecessary biopsies and negative treatment side effects, outweigh the benefits of finding and managing the disease early (Martin et al., JAMA 2018; 319:883; FIGS. 3 and 4).

Consequently, there is a great need for screening tests that are able to identify individuals with aggressive cancers, such as prostate cancer, prior to the exhibition of symptoms, as well a need to monitor disease progress and drug resistance once prostate cancer is diagnosed.

Similarly, with other cancers, there is a vital need to identify biomarkers, metabolite signatures and molecular networks that can provide early diagnosis and assess cancer and treatment status and future prognosis.

### SUMMARY

Experiments described herein utilized metabolomic/lipidomic analyses to discover plasma (e.g., lipid) biomarkers that reveal mechanisms leading to early detection of potentially aggressive cancers. These biomarkers also find use in determining the aggressiveness and drug resistance of existing cancers. Four separate lipid classes, as well as a pathway within one of the classes, where the metabolites in that pathway appear in plasma and are strongly associated and predictive of aggressive prostate cancer, are described herein.

For example, in some embodiments, provided herein is a method of predicting the risk of developing cancer (e.g., prostate cancer), providing a diagnosis of cancer (e.g., prostate cancer), providing a prognosis of cancer (e.g., prostate cancer) being aggressive, or monitoring treatment of cancer (e.g., prostate cancer), comprising: a) determining the presence, absence, or level of one or more (e.g., 1, 2, 3, 4, 5, 6, 7, 8, 9, 10, 15, 20, 25, 30, or all) metabolites selected from, for example, those described in Table 1 in a sample (e.g., tissue, blood or blood product (e.g., plasma) from a subject; and b) predicting the risk of developing cancer (e.g., prostate cancer), providing a diagnosis of cancer (e.g., prostate cancer), providing a prognosis of cancer (e.g., prostate cancer) being aggressive, or monitoring treatment of cancer (e.g., prostate cancer) based on said presence, absence, or level of the metabolites. In some embodiments, determining the presence, absence, or level of the metabolite comprises a chromatography and/or spectrometry method. In some embodiments, an increased level of said metabolite is indicative of an increased risk of aggressive cancer (e.g., prostate cancer), an increased likelihood of developing cancer (e.g., prostate cancer), a poor response to a treatment for cancer (e.g., prostate cancer), or resistance to a treatment for cancer (e.g., prostate cancer).

The present disclosure is not limited to a particular cancer. In some embodiments, the cancer is prostate cancer. Additional cancers include, but are not limited to, lung (e.g., squamous cell lung cancer), uterine, ovarian, melanoma, esophageal cancer, stomach cancer, etc.

The present disclosure is not limited to particular metabolites. In some exemplary embodiments, the metabolite is a ceramide (e.g., tetrahexosylceramide and/or a trihexosylceramide). In some embodiments, the metabolite is one or more (e.g., 1, 2, 3, 4, 5, 6, 7, 8, 9, 10, 11, 12, 13, 14, or 15 or 1 to 15, 2 to 15, 3 to 15, 4 to 15, 5 to 15, etc.) of tetrahexosylceramide, trihexosylceramide, ceramide (d18:1/22:0), SM(d18:0/24:1), cholesterol, cer(d18:1/24:1), C46H800, TG(14:1/18:1/18:1), lathosterol, C40H75N3O2, PC(20:0/14:0), cer(d18:1/23:0), PC(P-18:1/14:0), PE(P-18:0/22:0), or C16H62N32O29S8 (e.g., tetrahexosylceramide (d18:1/16:0), SM(d18:0/24:1), trihexosylceramide (d18:1/16:0), cer(d18:1/24:1), and ceramide(d18:1/22:0); tetrahexosylceramide (d18:1/16:0), SM(d18:0/24:1), trihexosylceramide (d18:1/16:0), and cer(d18:1/24:1); or tetrahexosylceramide (d18:1/16:0), SM(d18:0/24:1), and trihexosylceramide (d18:1/16:0)). In some embodiments, the metabolite is one or more (e.g., 1, 2, 3, 4, 5, 6, 7, 8, 9, 10, 11, 12, 13, 14, 15, 20, 25, 30, or 32 or more (e.g., 1 to 20, 5 to 20, 2 to 10, 2 to 15, 2 to 30) of tetrahexosylceramide, (d18:1/16:0), sphingomyelin (d18:1/24:1), trihexosylceramide (d18:1/16:0), ceramide (d18:1/24:1), ceramide (d18:1/22:0), triacylglyceride (14:1/18:1/18:1), lathosterol, heptacosanoic acid, phosphatidylcholine (20:0/14:0), ceramide, phosphatidylethanolamine (d14:2/18:1), sphingomyelin (d18:1/22:0), mulberrofuran E, Ceramide (d18:1/23:0), phosphatidylethanolamine (P-18:0/22:0), cholesterol, glucosylceramide (d18:1/16:0), ceramide (d18:2/23:0), phosphatidylcholine (P-18:1/14:0), triacylglyceride (14:1/14:1/20:1), phosphatidylcholine (18:0/14:0), phosphatidylserine (0-20:0/18:4), glucosylceramide (d18:1/16:0), phosphatidylcholine (A-16:0/16:0), ganglioside GM3 (d18:1/16:0), phosphatidylcholine (P-16:0/16:0), phosphatidylserine (P-20:0/22:4), phosphatidylethanolamine (24:0/P-18:1), ceramide (d18:0/24:1), alpha-Tocopherolquinone, ceramide 1-phosphate (d18:1/26:1), (S)-Abscisic acid, or lactosylceramide (d18:1/16:0).

The present disclosure is not limited to particular subject populations. Examples include, but are not limited to, a subject diagnosed with cancer, a subject not diagnosed with cancer, and a subject undergoing treatment for cancer. In some embodiments, the subject is a man of African or European descent.

In additional embodiments, the method comprises diagnosing or determining the risk of aggressive cancer or determining the effectiveness of a treatment for cancer.

The present disclosure is not limited to particular prostate cancer treatments. Examples include, but are not limited to, surgery, radiation, chemotherapy, hormone blocking therapy, or a combination thereof.

Further embodiments provide determining a treatment course of action and/or administering a treatment for cancer.

Also provided herein is a method of treating cancer (e.g., prostate cancer), comprising: a) determining the presence, absence, or level of one or more metabolites selected from those described in Table 1 in a sample from a subject; b) providing a recommended treatment based on presence, absence, or level of the metabolites; and c) administering the treatment to said subject. In some embodiments, the subject is currently undergoing a treatment for cancer. In some embodiments, the treatment is continued, discontinued, or altered based on the presence, absence, or level of the metabolites.

Yet other embodiments provide a kit for predicting the risk of developing cancer, providing a diagnosis of cancer, providing a prognosis of cancer (e.g., prostate cancer) being aggressive, or monitoring treatment of cancer (e.g., prostate cancer), comprising: reagents for determining the presence, absence, or level of one or more metabolites selected from those described in Table 1 in a sample from a subject.

Additional embodiments provide a method of predicting the risk of developing cancer, providing a diagnosis of cancer, providing a prognosis of cancer being aggressive, or monitoring treatment of cancer, comprising: a) determining the presence of a copy number variation (e.g., a genomic deletion and/or genomic amplification) in one or more (e.g., 1, 2, 3, 4, 5, 6, 7, 8, 9, 10, 1 to 10, 2 to 10, 3 to 10, 4 to 10, 5 to 10, 6 to 10, 7 to 10, 8 to 10, 9 to 10, etc.) genes selected from, for example, UGCG, B4GALT1, B4GALT5, A4GALT, B3GALTN1, GBA, GLB1, GLA, HEXA, and HEXB in a sample from a subject; and b) predicting the risk of developing cancer, providing a diagnosis of cancer, providing a prognosis of cancer being aggressive, or monitoring treatment of cancer based on the presence of the copy number variation.

Certain embodiments provide a method of identifying copy number variation, comprising: determining the presence of a copy number variation in one or more (e.g., 1, 2, 3, 4, 5, 6, 7, 8, 9, 10, 1 to 10, 2 to 10, 3 to 10, 4 to 10, 5 to 10, 6 to 10, 7 to 10, 8 to 10, 9 to 10, etc.) genes selected from, for example, UGCG, B4GALT1, B4GALT5, A4GALT, B3GALTN1, GBA, GLB1, GLA, HEXA, and HEXB in a sample from a subject.

Additional embodiments are described herein.

### DESCRIPTION OF THE FIGURES

FIG. 1 shows an overview of Sphingolipid Metabolism and Biology. SMS: sphingomyelin synthase; SMASE: sphingomyelinase; GCS: glucosylceramide synthase; B4GALT: beta-1,4-galctosyltransferase 1; A4GALT: lactosylceramide 4-alpha-galactosyltransferase; B3GALT: beta-1,3-galctosyltransferase 1; HEXA/HEXB: hexosaminidase alpha/beta; αGALA: alpha-galactosidase; GALC: galactosylcerbrosidase; GBA: glucosylceramidase.

FIG. 2 shows plasma lipid association with aggressiveness in the North Carolina-Louisiana Prostate Cancer Project. A) Metabolomic analysis revealed 38 plasma lipids were associated significantly with aggressiveness after FDR correction. B) European-American and African-American men in this cohort shared 11 common lipid biomarkers, which included eight sphingolipids that were associated with prostate cancer aggressiveness.

FIG. 3 shows sphingolipid association with prostate cancer aggressiveness. Trihexosylceramide, and tetrahexosylceramide, were elevated significantly in both intermediate- and high-aggressive prostate cancer samples. Glucosylceramide and lactosylceramide were elevated significantly in intermediate-aggressive prostate cancer samples.

FIG. 4 shows receiver operating characteristic (ROC analysis) for sphingolipid association with prostate cancer aggressiveness. The top 3 most intense sphingolipids [tetrahexosylceramide (d18:1/16:0), sphingomyelin (d18:0/24:1), and trihexosylceramide (d18:1/16:0)], top 4 [top 3 plus ceramide (d18:1/24:1)] and top 5 [top 4 plus ceramide (d18:1/22:0)] sphingolipids were analyzed by ROC for association with aggressiveness.

FIG. 5 shows analysis of changes in hexosylceramide metabolic genes from the Cancer Genome Atlas (TCGA). TCGA data were analyzed across cancers for alterations to hexosylceramide genes (A), and by anabolic and catabolic genes in prostate cancer (B). HEXB gene alterations examined across multiple online prostate cancer data sets demonstrated alteration frequencies of up to 10%.

FIG. 6 shows Table 1. Plasma metabolites identified significantly associated with PCA aggressiveness include sphingolipids, phospholipids, triglycerides, unesterified fatty acids, cholesterol/lathosterol.

FIG. 7 shows partial least squares-discriminant analysis (PLS-DA). Abbreviations: ceramide (Cer), sphingomyelin (SM), phosphotidylcholine (PC), phosphatidylethanolamine (PE) and triacylglycerol (TAG).

FIG. 8 shows lipids associated with prostate cancer aggressiveness. A) ROC analysis of lipid metabolites in prostate cancer plasmas were categorized according to lipid class, demonstrating a prevalence of A) triglyceride-related compounds, B) cholesterol-related compounds, C) saturated phospholipid-related compounds, and D) sphingolipids.

FIG. 9 shows ROC analysis of PSA vs aggressiveness. PSA was analyzed by ROC for association with aggressiveness.

FIG. 10 shows ROC analysis of ceramide-containing compounds vs Grade Group 1 verses Grade Groups 2-5.

FIG. 11 shows targeted metabolomics. Main Panel: Absolute quantitation of complex glycosphingolipids. Upper Left: calibration curves with an average R\hat{ }2 of 0.999. Upper Right: Lower limits of detection.

FIG. 12 shows quantitation of major glycosphingolipid biomarkers from 100μl plasma.

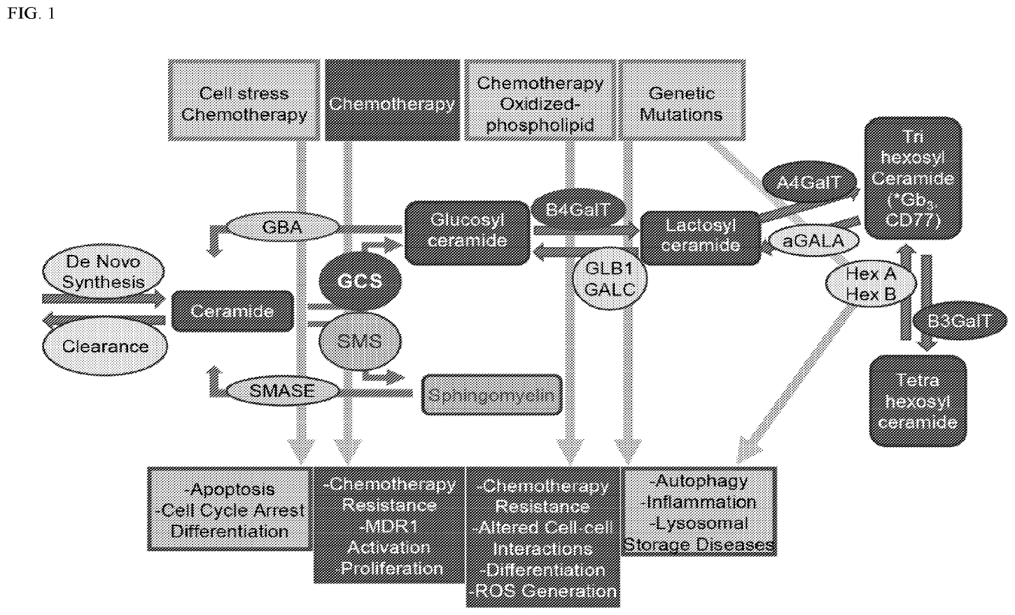

### DEFINITIONS

To facilitate an understanding of the present invention, a number of terms and phrases are defined below:

As used herein, the terms “detect”, “detecting” or “detection” may describe either the general act of discovering or discerning or the specific observation of a metabolite.

As used herein, the term “subject” refers to any organisms that are screened using the methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer markers, including but not limited to, those described herein

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, tumors, (e.g., biopsy samples), cells, and gases. Biological samples include blood products, such as plasma, serum and the like. Such examples are not however to be construed as limiting the sample types applicable to the present invention.

A “reference level” of a metabolite means a level of the metabolite that is indicative of a particular disease state, phenotype, or lack thereof, as well as combinations of disease states, phenotypes, or lack thereof. A “positive” reference level of a metabolite means a level that is indicative of a particular disease state or phenotype. A “negative” reference level of a metabolite means a level that is indicative of a lack of a particular disease state or phenotype. For example, a “prostate cancer-positive reference level” of a metabolite means a level of a metabolite that is indicative of a positive diagnosis of prostate cancer in a subject, and a “prostate cancer-negative reference level” of a metabolite means a level of a metabolite that is indicative of a negative diagnosis of prostate cancer in a subject. A “reference level” of a metabolite may be an absolute or relative amount or concentration of the metabolite, a presence or absence of the metabolite, a range of amount or concentration of the metabolite, a minimum and/or maximum amount or concentration of the metabolite, a mean amount or concentration of the metabolite, and/or a median amount or concentration of the metabolite; and, in addition, “reference levels” of combinations of metabolites may also be ratios of absolute or relative amounts or concentrations of two or more metabolites with respect to each other. Appropriate positive and negative reference levels of metabolites for a particular disease state, phenotype, or lack thereof may be determined by measuring levels of desired metabolites in one or more appropriate subjects, and such reference levels may be tailored to specific populations of subjects (e.g., a reference level may be age-matched so that comparisons may be made between metabolite levels in samples from subjects of a certain age and reference levels for a particular disease state, phenotype, or lack thereof in a certain age group). Such reference levels may also be tailored to specific techniques that are used to measure levels of metabolites in biological samples (e.g., LC-MS, GC-MS, etc.), where the levels of metabolites may differ based on the specific technique that is used.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

“Mass Spectrometry” (MS) is a technique for measuring and analyzing molecules that involves fragmenting a target molecule, then analyzing the fragments, based on their mass/charge ratios, to produce a mass spectrum that serves as a “molecular fingerprint”. Determining the mass/charge ratio of an object is done through means of determining the wavelengths at which electromagnetic energy is absorbed by that object. There are several commonly used methods to determine the mass to charge ration of an ion, some measuring the interaction of the ion trajectory with electromagnetic waves, others measuring the time an ion takes to travel a given distance, or a combination of both. The data from these fragment mass measurements can be searched against databases to obtain definitive identifications of target molecules. Mass spectrometry is also widely used in other areas of chemistry, like petrochemistry or pharmaceutical quality control, among many others.

The term “metabolism” refers to the chemical changes that occur within the tissues of an organism, including “anabolism” and “catabolism”. Anabolism refers to biosynthesis or the buildup of molecules and catabolism refers to the breakdown of molecules.

A “metabolite” is an intermediate or product resulting from metabolism. Metabolites are often referred to as “small molecules”.

The term “metabolomics” refers to the study of cellular metabolites.

As used herein, the term “clinical failure” refers to a negative outcome following prostatectomy. Examples of outcomes associated with clinical failure include, but are not limited to, an increase in PSA levels (e.g., an increase of at least 0.2 ng ml⁻¹) or recurrence of disease (e.g., metastatic prostate cancer) after prostatectomy.

As used herein, the term “multiplex” refers to the detection of more than one substance (e.g., analyte, metabolite, compound) in a sample simultaneously.

### DETAILED DESCRIPTION OF THE INVENTION

The uncertainty of PSA as an early detection tool and the wide variability in outcomes for certain Grade Groups warrants the search for better biomarkers to determine prostate cancer aggressiveness. Numerous studies have examined the impact of various lipid classes and molecular species on prostate cancer development and progression. Growth and survival have been associated with up-regulated lipogenesis (“lipogenic and lypolytic phenotypes”) (Menendez J A, Lupu R: Fatty acid synthase and the lipogenic phenotype in cancer pathogenesis. Nat Rev Cancer 7:763-77, 2007; Zadra G L, M.: Metabolic vulnerabilities of prostate cancer: diagnostic and therapeutic opportunities. Cold Spring Harb Perspect Med., 2017; Medes G, Thomas A, Weinhouse S: Metabolism of neoplastic tissue. IV. A study of lipid synthesis in neoplastic tissue slices in vitro. Cancer Res 13:27-9, 1953; Migita T, Ruiz S, Fornari A, et al: Fatty acid synthase: a metabolic enzyme and candidate oncogene in prostate cancer. J Natl Cancer Inst 101:519-32, 2009; Angeles T S, Hudkins R L: Recent advances in targeting the fatty acid biosynthetic pathway using fatty acid synthase inhibitors. Expert Opin Drug Discov 11:1187-1199, 2016; Zaidi N, Lupien L, Kuemmerle N B, et al: Lipogenesis and lipolysis: the pathways exploited by the cancer cells to acquire fatty acids. Prog Lipid Res 52:585-9, 2013; Kuemmerle N B, Rysman E, Lombardo P S, et al: Lipoprotein lipase links dietary fat to solid tumor cell proliferation. Mol Cancer Ther 10:427-36, 2011). Studies of circulating lipids in prostate cancer have focused on unsaturated fatty acids, cholesterol, phospholipids, and lyso-phosphlipids (Bull C J, Bonilla C, Holly J M, et al: Blood lipids and prostate cancer: a Mendelian randomization analysis. Cancer Med 5:1125-36, 2016; Crowe F L, Appleby P N, Travis R C, et al: Circulating fatty acids and prostate cancer risk: individual participant meta-analysis of prospective studies. J Natl Cancer Inst 106, 2014; Zhou X, Mao J, Ai J, et al: Identification of plasma lipid biomarkers for prostate cancer by lipidomics and bioinformatics. PLoS One 7:e48889, 2012; Kdadra M, Hockner S, Leung H, et al: Metabolomics Biomarkers of Prostate Cancer: A Systematic Review. Diagnostics (Basel) 9, 2019).

Sphingolipids are understood to have a primarily structural role in cellular membranes. Critical roles in cancer signaling and biology are now being recognized for key sphingolipids, which include sphingosine 1-phosphate (S1P), ceramide, ceramide 1-phosphate (C1P), and as a metabolic hub for the synthesis of several classes of sphingolipids, which include sphingomyelin (SM), C1P, and glycosphingolipids. SM is generated by the addition of phosphocholine to ceramide, while glucosylceramide is generated by the addition of glucose to ceramide via glucosylceramide synthase (GCS). Glucosylceramide is the substrate for complex glycosphingolipids generated via the sequential addition of sugar moieties to produce lactosylceramide and more complex glycosphingolipids, such as tri- and tetra-hexosylceramides (Russo D, Capolupo L, Loomba J S, et al: Glycosphingolipid metabolism in cell fate specification. J Cell Sci 131, 2018). Studies in the late 1990s revealed that in vitro accumulation of glucosylceramides was associated strongly with multidrug resistance (MDR) in numerous cancer cell lines (Gouaze-Andersson V, Cabot M C: Glycosphingolipids and drug resistance. Biochim Biophys Acta 1758:2096-103, 2006; Lucci A, Cho W I, Han T Y, et al: Glucosylceramide: a marker for multiple-drug resistant cancers. Anticancer Res 18:475-80, 1998; Lavie Y, Cao H, Bursten S L, et al: Accumulation of glucosylceramides in multidrug-resistant cancer cells. J Biol Chem 271:19530-6, 1996; Veldman R J, Klappe K, Hinrichs J, et al: Altered sphingolipid metabolism in multidrug-resistant ovarian cancer cells is due to uncoupling of glycolipid biosynthesis in the Golgi apparatus. FASEB J 16:1111-3, 2002). More recent studies indicated changes in differential expression of sphingolipids, specifically glycosphingolipids and their metabolic enzymes, may play critical roles in initiation and malignant transformation of numerous cancers (Zhuo D, Li X, Guan F: Biological Roles of Aberrantly Expressed Glycosphingolipids and Related Enzymes in Human Cancer Development and Progression. Front Physiol 9:466, 2018).

Metabolomics and other unbiased approaches are beginning to fill critical roles in defining precipitating events in cancer development, identifying molecular indicators of the cancer trajectory, and predicting targeted treatment strategies that prolong lifespan and improve overall quality of life. Two recent reviews examined the potential role of metabolites and molecular signatures in blood, urine, (tumor) tissues, or extracellular vesicles in prostate cancer (Kdadra M, Hockner S, Leung H, et al: Metabolomics Biomarkers of Prostate Cancer: A Systematic Review. Diagnostics (Basel) 9, 2019; Gomez-Cebrian N, Rojas-Benedicto A, Albors-Vaguer A, et al: Metabolomics Contributions to the Discovery of Prostate Cancer Biomarkers. Metabolites 9, 2019). Several metabolites have been identified that may aid prostate cancer early detection and augment the Gleason scoring system, to differentiate lethal from indolent prostate cancer. There is a clear need for more definitive metabolomic studies that focus on molecular networks (pathways) underpinning prostate cancer occurrence and aggressiveness.

Accordingly, provided herein is a glucosylceramide molecular network that serves as a diagnostic for cancer as exemplified by prostate cancer and plays a role in determining aggressiveness of prostate cancer in men (e.g., men of African and European descent) and other cancers in a variety of patient populations. The present disclosure is exemplified with prostate cancer. However, the described compositions and methods find use in the diagnosis, characterization, and prognosis of a variety of cancer (e.g., including but not limited to, prostate, lung (e.g., squamous cell lung cancer), uterine, ovarian, melanoma, esophageal cancer, stomach cancer, etc.).

For example, in some embodiments, provides herein is a method of predicting the risk of developing cancer, providing a diagnosis of cancer, providing a prognosis of cancer being aggressive, or monitoring treatment of cancer, comprising: a) determining the presence, absence, or level of one or more (e.g., 1, 2, 3, 4, 5, 6, 7, 8, 9, 10, 15, 20, 25, 30, or all) metabolites or genes involved in a glycosphingolipid metabolism pathway selected from, for example, those described in Table 1 in a sample (e.g., tissue, blood or blood product (e.g., plasma) from a subject; and b) predicting the risk of developing cancer, providing a diagnosis of cancer, providing a prognosis of cancer being aggressive, or monitoring treatment of cancer based on said presence, absence, or level of the metabolites. In some embodiments, the determining the presence, absence, or level of the metabolite comprises a chromatography and/or spectrometry method. In some embodiments, an increased level of said metabolite is indicative of an increased risk of aggressive cancer, an increased likelihood of developing cancer, a poor response to a treatment for cancer, or resistance to a treatment for cancer.

The present disclosure is not limited to particular metabolites. In some exemplary embodiments, the metabolite is a ceramide (e.g., tetrahexosylceramide and/or a trihexosylceramide). In some embodiments, the metabolite is one or more of tetrahexosylceramide, trihexosylceramide, ceramide (d18:1/22:0), SM(d18:0/24:1), cholesterol, cer(d18:1/24:1), C46H800, TG(14:1/18:1/18:1), lathosterol, C40H75N3O2, PC(20:0/14:0), cer(d18:1/23:0), PC(P-18:1/14:0), PE(P-18:0/22:0), or C16H62N32029S8 (e.g., tetrahexosylceramide (d18:1/16:0), SM(d18:0/24:1), trihexosylceramide (d18:1/16:0), cer(d18:1/24:1), and ceramide(d18:1/22:0); tetrahexosylceramide (d18:1/16:0), SM(d18:0/24:1), trihexosylceramide (d18:1/16:0), and cer(d18:1/24:1); or tetrahexosylceramide (d18:1/16:0), SM(d18:0/24:1), and trihexosylceramide (d18:1/16:0)). In some embodiments, the metabolite is one or more (e.g., 1, 2, 3, 4, 5, 6, 7, 8, 9, 10, 11, 12, 13, 14, 15, 20, 25, 30, or 32 or more (e.g., 1 to 20, 5 to 20, 2 to 10, 2 to 15, 2 to 30) of tetrahexosylceramide, (d18:1/16:0), sphingomyelin (d18:1/24:1), trihexosylceramide (d18:1/16:0), ceramide (d18:1/24:1), ceramide (d18:1/22:0), triacylglyceride (14:1/18:1/18:1), lathosterol, heptacosanoic acid, phosphatidylcholine (20:0/14:0), ceramide, phosphatidylethanolamine (d14:2/18:1), sphingomyelin (d18:1/22:0), mulberrofuran E, Ceramide (d18:1/23:0), phosphatidylethanolamine (P-18:0/22:0), cholesterol, glucosylceramide (d18:1/16:0), ceramide (d18:2/23:0), phosphatidylcholine (P-18:1/14:0), triacylglyceride (14:1/14:1/20:1), phosphatidylcholine (18:0/14:0), phosphatidylserine (0-20:0/18:4), glucosylceramide (d18:1/16:0), phosphatidylcholine (A-16:0/16:0), ganglioside GM3 (d18:1/16:0), phosphatidylcholine (P-16:0/16:0), phosphatidylserine (P-20:0/22:4), phosphatidylethanolamine (24:0/P-18:1), ceramide (d18:0/24:1), alpha-Tocopherolquinone, ceramide 1-phosphate (d18:1/26:1), (S)-Abscisic acid, or lactosylceramide (d18:1/16:0).

In some embodiments, instead of or in addition to the metabolites described herein, the presence of a copy number variation (e.g., genomic amplification or genomic deletion) or altered level of expression of one or more (e.g., 1, 2, 3, 4, 5, 6, 7, 8, 9, 10, 1 to 10, 2 to 10, 3 to 10, 4 to 10, 5 to 10, 6 to 10, 7 to 10, 8 to 10, 9 to 10, etc.) genes associated with glucosylceramide synthesis and metabolism selected from, for example, UGCG, B4GALT1, B4GALT5, A4GALT, B3GALTN1, GBA, GLB1, GLA, HEXA, and HEXB are detected

The present disclosure is not limited to particular subject populations. Examples include, but are not limited to, a subject diagnosed with cancer, a subject not diagnosed with cancer, and a subject undergoing treatment for cancer.

In additional embodiments, the method comprises diagnosing or determining the risk of aggressive cancer or determining the effectiveness of a treatment for cancer.

The present disclosure is not limited to particular prostate cancer treatments. Examples include, but are not limited to, surgery, radiation, chemotherapy, hormone blocking therapy, or a combination thereof.

Further embodiments provide determining a treatment course of action and/or administering a treatment for cancer.

Also provided herein is a method of treating cancer, comprising: a) determining the presence, absence, or level of one or more metabolites selected from those described in Table 1 in a sample from a subject; b) providing a recommended treatment based on presence, absence, or level of the metabolites; and c) administering the treatment to said subject. In some embodiments, the subject is currently undergoing a treatment for cancer. In some embodiments, the treatment is continued, discontinued, or altered based on the presence, absence, or level of the metabolites.

Any patient sample suspected of containing cancer-specific metabolites is tested according to the methods described herein. By way of non-limiting examples, the sample may be tissue (e.g., a prostate sample or post-surgical tissue), blood, urine, or a fraction thereof (e.g., plasma, serum, urine supernatant, urine cell pellet, urine sediment, or prostate cells). In some embodiments, the sample is a tissue sample obtained from a biopsy or following surgery (e.g., prostate biopsy). A urine sample is preferably collected immediately following an attentive digital rectal examination (DRE), which causes prostate cells from the prostate gland to shed into the urinary tract.

In some embodiments, the patient sample undergoes preliminary processing designed to isolate or enrich the sample for cancer specific metabolites or cells that contain cancer specific metabolites. A variety of techniques may be used for this purpose, including but not limited: centrifugation; immunocapture; and cell lysis.

Metabolites may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized. In other embodiments, metabolites (e.g., biomarkers and derivatives thereof) are detected using optical imaging techniques such as magnetic resonance spectroscopy (MRS), magnetic resonance imaging (MM), CAT scans, ultra sound, MS-based tissue imaging or X-ray detection methods (e.g., energy dispersive x-ray fluorescence detection).

Any suitable method may be used to analyze the biological sample in order to determine the presence, absence or level(s) of the one or more metabolites in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

The levels of one or more of the recited metabolites may be determined in the methods of the present invention. For example, the level(s) of one metabolites, two or more metabolites, three or more metabolites, four or more metabolites, five or more metabolites, six or more metabolites, seven or more metabolites, eight or more metabolites, nine or more metabolites, ten or more metabolites, etc., including a combination of some or all of the metabolites described herein may be determined and used in such methods. Determining levels of combinations of the metabolites may allow greater sensitivity and specificity in the methods, such as diagnosing prostate cancer and aiding in the diagnosis of prostate cancer, and may allow better differentiation or characterization of prostate cancer from other prostate disorders, or determining aggressiveness of prostate cancer or other cancers that may have similar or overlapping metabolites to prostate cancer (as compared to a subject not having prostate cancer). For example, ratios of the levels of certain metabolites in biological samples may allow greater sensitivity and specificity in diagnosing prostate cancer and aiding in the diagnosis of prostate cancer and allow better differentiation or characterization of prostate cancer from other cancers or other disorders of the prostate that may have similar or overlapping metabolites to prostate cancer (as compared to a subject not having prostate cancer).

Copy number variations (CNVs) are detected using any suitable method. In some embodiments, CNV detection methods utilize hybridization methods. In some embodiments, the hybridization is a fluorescence in situ hybridization (FISH) method. FISH is traditionally performed using fluorescently labeled DNA probes generated from known large chromosomal regions cloned into bacterial artificial chromosomes (BAC). These fluorescently labeled DNA probes are complementary to intended targets and hybridize. FISH is generally a single-cell technique that assesses the number of copies of targets present in every cell. Thus, deletions and amplifications result in the loss or gain of signal compared to control probes that are typically designed to centromeric regions. Additional methods include array based methods and sequencing methods (See e.g., Li et al., Physiol Genomics. 2013 January; 45(1): 1-16; herein incorporated by reference in its entirety).

In some embodiments, a computer-based analysis program is used to translate the raw data generated by the detection assay (e.g., the presence, absence, or amount of a cancer specific metabolite) into data of predictive value for a clinician. The clinician can access the predictive data using any suitable means. Thus, in some embodiments, the present invention provides the further benefit that the clinician, who is not likely to be trained in metabolite analysis, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a blood, urine or plasma sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., metabolic profile), specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of cancer being present, aggressiveness of the cancer, risk of developing cancer in the future) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

When the amount(s) or level(s) of the one or more metabolites in the sample are determined, the amount(s) or level(s) may be compared to cancer metabolite-reference levels, such as cancer-positive and/or cancer-negative reference levels to aid in diagnosing or to diagnose whether the subject has cancer. Levels of the one or more metabolites in a sample corresponding to the cancer-positive reference levels (e.g., levels that are the same as the reference levels, substantially the same as the reference levels, above and/or below the minimum and/or maximum of the reference levels, and/or within the range of the reference levels) are indicative of a diagnosis, risk, or prognosis of cancer in the subject. Levels of the one or more metabolites in a sample corresponding to the cancer-negative reference levels (e.g., levels that are the same as the reference levels, substantially the same as the reference levels, above and/or below the minimum and/or maximum of the reference levels, and/or within the range of the reference levels) are indicative of a diagnosis of no cancer in the subject. In addition, levels of the one or more metabolites that are differentially present (especially at a level that is statistically significant) in the sample as compared to cancer-negative reference levels are indicative of a diagnosis of cancer in the subject. Levels of the one or more metabolites that are differentially present (especially at a level that is statistically significant) in the sample as compared to cancer-positive reference levels are indicative of a diagnosis of no cancer in the subject. In some embodiments, quantitative reference levels for a specific diagnosis or prognosis are determined and utilized to provide a risk assessment, diagnosis, prognosis, or treatment.

The level(s) of the one or more metabolites are compared to cancer-positive and/or prostate cancer-negative reference levels using various techniques, including a simple comparison (e.g., a manual comparison) of the level(s) of the one or more metabolites in the biological sample to cancer-positive and/or cancer-negative reference levels. The level(s) of the one or more metabolites in the biological sample may also be compared to cancer-positive and/or cancer-negative reference levels using one or more statistical analyses (e.g., t-test, Welch's T-test, Wilcoxon's rank sum test, random forest).

Compositions for use (e.g., sufficient for, necessary for, or useful for) in the diagnostic, research or screening methods of some embodiments of the present invention include reagents necessary, sufficient or useful for detecting the presence or absence or levels of cancer specific metabolites. Any of these compositions, alone or in combination with other compositions of the present invention, may be provided in the form of a kit. Kits may further comprise appropriate controls and/or detection reagents.

Embodiments of the present invention provide for multiplex or panel assays that simultaneously detect one or more of the markers of the present invention (e.g., those described in Table 1, alone or in combination with additional cancer markers. For example, in some embodiments, panel or combination assays are provided that detected 2 or more, 3 or more, 4 or more, 5 or more, 6 or more, 7 or more, 8 or more, 9 or more, 10 or more, 15 or more, or 20 or more markers in a single assay. In some embodiments, assays are automated or high throughput.

In some embodiments, additional cancer markers are included in multiplex or panel assays. Markers are selected for their predictive value alone or in combination with the metabolic markers described herein. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); and, those disclosed in U.S. Pat. Nos. 5,854,206 and 6,034,218, and U.S. Publication No. 20030175736, each of which is herein incorporated by reference in its entirety. Markers for other cancers, diseases, infections, and metabolic conditions are also contemplated for inclusion in a multiplex or panel format.

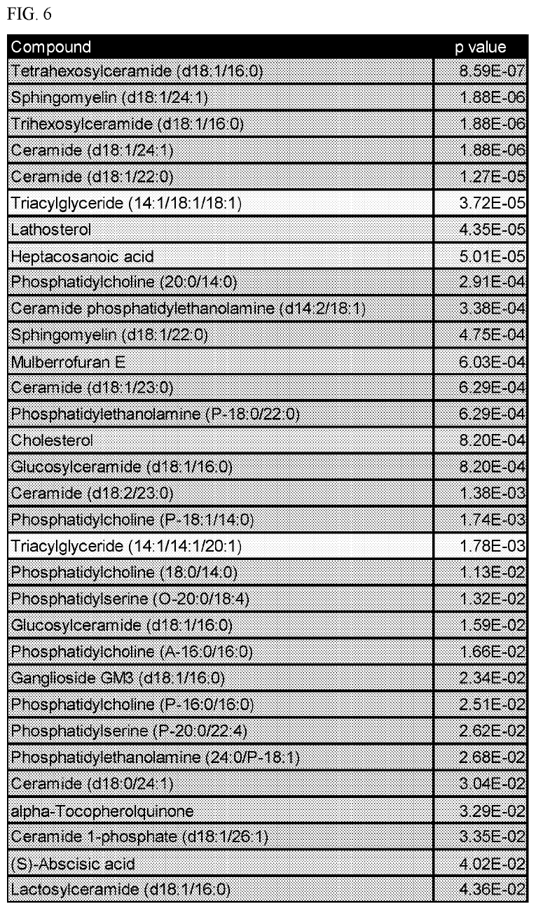

In some embodiments, additional markers are expression levels of genes related to the lipid markers described herein (See e.g., FIG. 6).

### EXPERIMENTAL

### Example 1

### Methods:

Cohort: The North Carolina Louisiana Prostate Cancer Project (PCaP) is a longitudinal study that includes self-reported African-American (AfAm) and European-American (EuAm) participants with a diagnosis of prostate cancer (Schroeder J C, Bensen J T, Su L J, et al: The North Carolina-Louisiana Prostate Cancer Project (PCaP): methods and design of a multidisciplinary population-based cohort study of racial differences in prostate cancer outcomes. Prostate 66:1162-76, 2006). Retrospective, de-identified plasma samples were analyzed from 80 African-American (AfAm) and 79 European-American (EuAm) men obtained prior to treatment for prostate cancer (Below Table) in accordance with a Wake Forest Health Sciences Investigational Review Board approved protocol.

### Demographics for the PCaP Cohort Examined.

Demographics (N = 159)

| | African | European |
|---|---|---|
| | American | American |
| Reported Race | 80 | 79 |

| | | | |
|---|---|---|---|
| Age at Diagnosis | Avg. (S.D.) | 62 (9) | 66 (8) |
| | ≤50 | 11 | 2 |
| | 51-55 | 9 | 8 |
| | 56-60 | 15 | 9 |
| | 61-65 | 14 | 19 |
| | 66-70 | 17 | 15 |
| | 71-75 | 12 | 15 |
| | >75 | 2 | 11 |

Measures of Severity

| | | | |
|---|---|---|---|
| Aggressiveness | Low | 27 | 30 |
| | Intermediate | 37 | 28 |
| | High | 16 | 21 |
| Grade Group | 1 | 29 | 31 |
| | 2 | 25 | 25 |
| | 3 | 15 | 8 |
| | 4 + 5 | 11 | 15 |

Main comparisons of interest: The correlation between levels of plasma metabolomic compounds (with a focus on the lipidome) and prostate cancer Aggressiveness was analyzed. Aggressiveness was classified based on clinical Grade Group, clinical stage, and PSA at diagnosis (Schroeder et al., supra) such that: (1) Aggressive=Grade Group 4 or 5, or PSA>20 ng/ml, or Grade Group 2 or 3, and stage cT3-cT4; (2) Non-Aggressive=Grade Group 1 and stage cT1-cT2, and PSA<10 ng/ml; and (3) Intermediate Aggressive=all other cases. Comparisons with biomarkers have been evaluated for the PCaP Aggressiveness score and for Grade Groups 1 vs 2-5.

Sample Preparation: 50 μL of each plasma sample were extracted with 190 μL of LCMS grade methanol and 10μL of SPLASH LipidoMIX Internal standard mixture (Avanti, Al). Samples were vortexed (4° C. for 30 minutes), centrifuged (13,000 RPM at 4° C. for 15 minutes), and supernatant evaporated under nitrogen gas before resuspension in 100 μL of LCMS grade methanol. Quality Control (QC) mixtures were created for plasma samples by pooling 10 μL from each sample.

UPLC-MS Analysis: 90 uL of extract was dried under nitrogen and suspended in 100 μL of toluene/methanol (3/2, v/v). 3 μL of extract was injected onto a Waters Acquity UPLC system in randomized order, and separated using a Waters Acquity UPLC CSH Phenyl Hexyl column (1.7 1.0×100 mm), using a gradient from solvent A (water, 0.1% formic acid) to solvent B (Acetonitrile, 0.1% formic acid). Injections were made in 100% A, held at 100% A for 1 min, ramped to 98% B over 12 minutes, held at 98% B for 3 minutes, and re-equilibrated utilizing a 200 μL/min flow rate. The column and samples were held at 65° C. and 6° C., respectively. Eluent was infused via electron spray ionization (ESI) source into a Waters Xevo G2 Q-TOF-MS in positive mode, scanning 50-2000 m/z at 0.2 seconds per scan, and alternated between MS (6 V collision energy) and MSE mode (15-30 V ramp). Calibration was performed using sodium iodide with 1 ppm mass accuracy. Capillary voltage was 2200 V, source temperature was 150° C., and nitrogen desolvation temperature was 350° C. with flow rate 800 L/hr. Annotations were assigned based on computational interpretation of MS signals. 18:1(d9) sphingomyelin (spiked into samples as Avanti SPLASH prior to extraction) was utilized for semi-quantitative assessment of ceramides and hexoslyceramides due to the similarity in structure, comparable ionization potential in positive ion mode, and similar retention time.

Data Analysis and Statistics: For each sample, raw data files were converted to cdf format, and matrix of molecular features defined by retention time and mass (m/z) was generated using XCMS software in R (Smith C A, Want E J, O'Maille G, et al: XCMS: processing mass spectrometry data for metabolite profiling using nonlinear peak alignment, matching, and identification. Anal Chem 78:779-87, 2006) for feature detection and alignment. The matchedFilter algorithm was used for GC-MS data and the centWave algorithm was used for LC-MS data. Features were grouped using RAMClustR (Broeckling C D, Afsar F A, Neumann S, et al: RAMClust: a novel feature clustering method enables spectral-matching-based annotation for metabolomics data. Anal Chem 86:6812-7, 2014), with normalization set to ‘TIC’ (total ion current). LC-MS data were annotated by searching against an in-house spectra and retention time database using RAMSearch. RAMClustR was used to call the findMain (Jaeger C, Meret M, Schmitt C A, et al: Compound annotation in liquid chromatography/high-resolution mass spectrometry based metabolomics: robust adduct ion determination as a prerequisite to structure prediction in electrospray ionization mass spectra. Rapid Commun Mass Spectrom 31:1261-1266, 2017) function from the interpretMSSpectrum package to infer the molecular weight of each LC-MS compound and annotate the mass signals. The complete MS spectrum and a truncated MSE spectrum were written to a .mat format for import to MSFinder (Tsugawa H, Kind T, Nakabayashi R, et al: Hydrogen Rearrangement Rules: Computational MS/MS Fragmentation and Structure Elucidation Using MS-FINDER Software. Anal Chem 88:7946-58, 2016). The MSE spectrum was truncated to only include masses with values less than the inferred M plus its isotopes, and the .mat file precursor ion is set to the M+H ion for the findMain inferred M value. These mat spectra were analyzed to determine the most probable molecular formula and structure. MSFinder was used to perform a spectral search against the MassBank database. All results were imported into R and a collective annotation was derived with prioritization of RAMSearch>MSFinder mssearch>MSFinder structure>MSFinder formula>findMain M. Annotation confidence was reported as described (Schroder F H, Hugosson J, Roobol M J, et al: Screening and prostate cancer mortality: results of the European Randomised Study of Screening for Prostate Cancer (ERSPC) at 13 years of follow-up. Lancet 384:2027-35, 2014). All R work was performed using R 3.3.1 (Andriole G L, Crawford E D, Grubb R L, 3rd, et al: Prostate cancer screening in the randomized Prostate, Lung, Colorectal, and Ovarian Cancer Screening Trial: mortality results after 13 years of follow-up. J Natl Cancer Inst 104:125-32, 2012). All statistical analysis and figure generation was performed in R. An unpaired Student's t-test with false discovery rate (FDR) correction was performed with FDR-corrected p-values<0.05 were considered significant in comparing AfAm- and EuAm-men.

### Results:

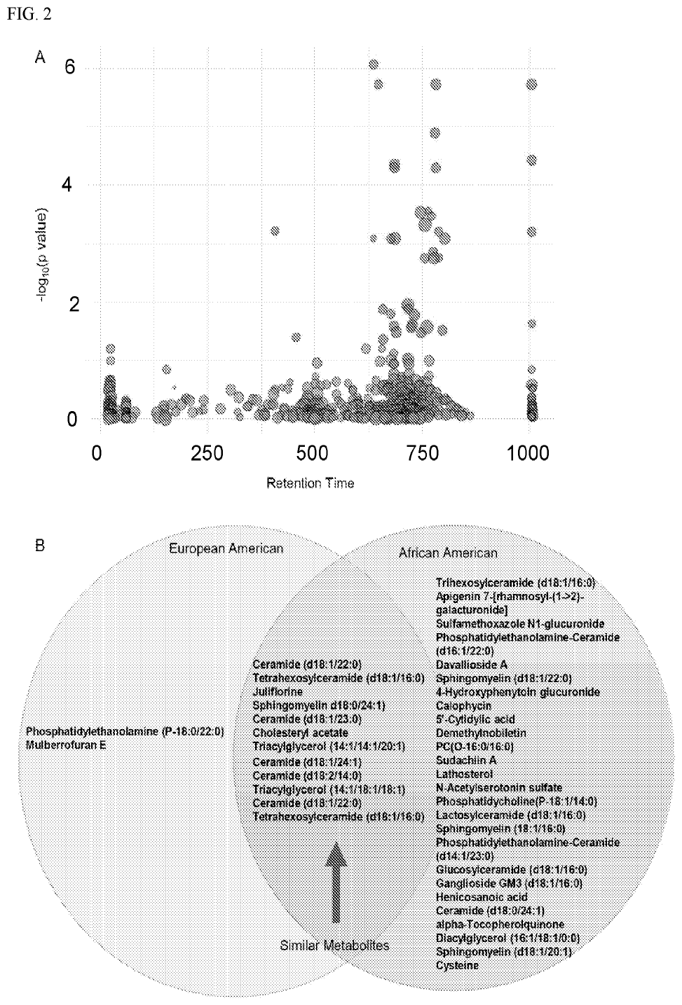

Untargeted metabolomic analyses were performed on plasma samples from 159 men with prostate cancer who had similar ages and severity distributions to determine if there was a common set of metabolites associated with prostate cancer aggressiveness. Initial analysis compared peak intensities of circulating small molecules/metabolites with tumor aggressiveness (Schroeder J C, Bensen J T, Su L J, et al: The North Carolina-Louisiana Prostate Cancer Project (PCaP): methods and design of a multidisciplinary population-based cohort study of racial differences in prostate cancer outcomes. Prostate 66:1162-76, 2006). Thirty-eight metabolites were associated significantly with aggressiveness (FIG. 2A and Table 1) after FDR correction. All but three of these metabolites were molecular species of five distinct lipid classes that included phospholipids, sphingolipids, triglycerides, unesterified fatty acids, and cholesterol/lathosterol. Fifteen of the molecular species most associated with prostate cancer aggressiveness were sphingolipids, including the top 5 by significance. (Table 1). Those with the strongest associations were sphingomyelins and glycosphingolipids, which included tetrahexosylceramide (d18:1/16:0) and trihexosylceramide (d18:1/16:0).

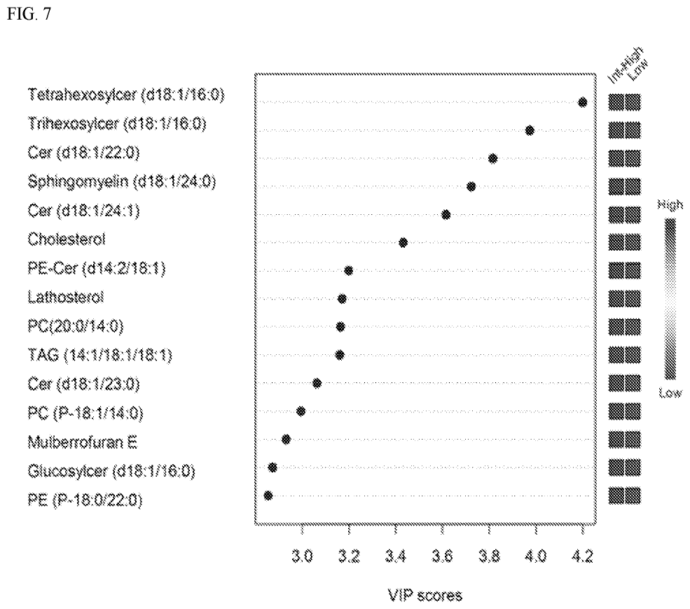

Metabolomic data from AfAm- and EuAm-men were analyzed separately to explore if there were race/ethnic specific metabolites associated with prostate cancer status (Grade Group 1 vs Grade Group 2-5). Thirty-eight metabolites were significant in AfAm-men compared to 12 metabolites in EuAm-men (FIG. 7). Of the metabolites observed in both groups, 10 were common, and these included previously described metabolites involved in glycosphingolipid synthesis. Of those exclusive to EuAm-men, 1 was a phospholipid containing saturated fatty acids at the sn-1 and sn-2 positions and one compound (tentatively annotated as Mulberrofuran E) typically found in fruits. Twenty additional metabolites were associated with prostate cancer severity in AfAm men, and these metabolites included numerous molecular species in the sphingolipid, phospholipid, and cholesterol metabolic networks.

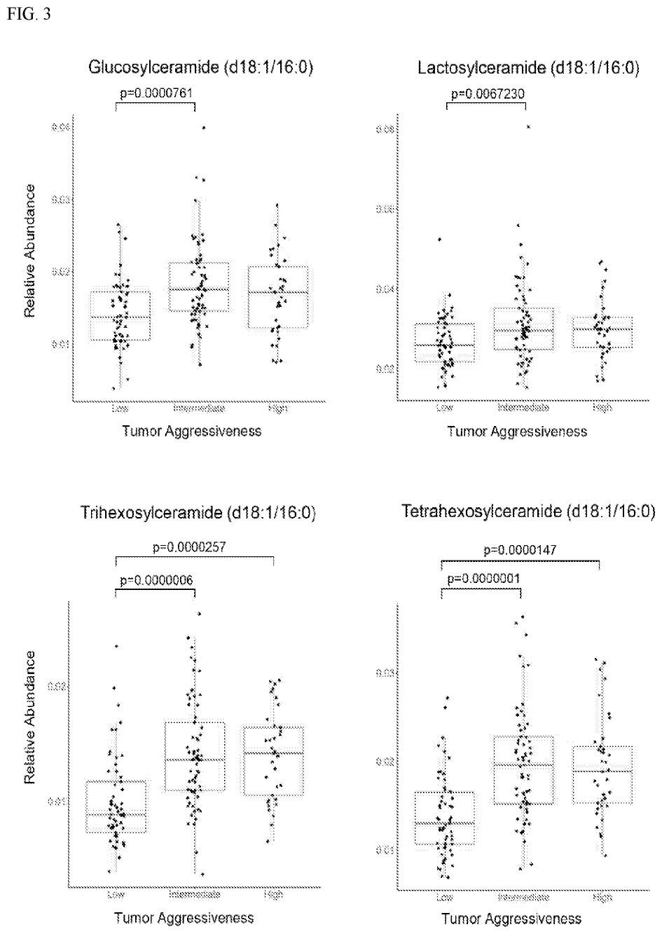

Index values of the Variable Importance in Projection (VIP) in Partial least squares-discriminant analysis (PLS-DA) were used to evaluate the capacity of individual molecules to distinguish low from intermediate-high aggressiveness. Similar to data shown in FIG. 2, PLS-DA of the entire data set identified the same molecular species of sphingomyelins and glycosphingolipids, with five of the top six having the most significant VIP scores (FIG. 7). Together, these data indicate that these sphingolipids play a role in prostate cancer aggressiveness and serve as meaningful biomarkers across racial/ethnic populations. Untargeted metabolomics were utilized initially as an unbiased approach to determine perturbations in molecular networks without an a priori metabolic hypothesis. The data provided by untargeted metabolomics is “compositional data” where individual components (or the signal intensity of individual metabolites) are a proportion of the whole (or total signal). Consequently, these data do not provide a quantitative measure of each metabolite. To overcome this limitation, further analyses were conducted using the internal standard mixture of deuterated lipids, which included a deuterated sphingomyelin molecular specie (N-oleoyl (d9)-D-erythro-sphingosylphosphorylcholine), to provide semi-quantitative measurements of sphingomyelins and glycosphingolipids. FIG. 3 illustrated the relative abundance of glucosylceramide, lactosylceramide, and tri- and tetra hexosylceramides in relation to prostate cancer aggressiveness (Schroeder et al., supra). These data revealed that the relative abundance of specific molecular species of hexoslyceramides were different in low versus intermediate or high aggressive prostate cancer.

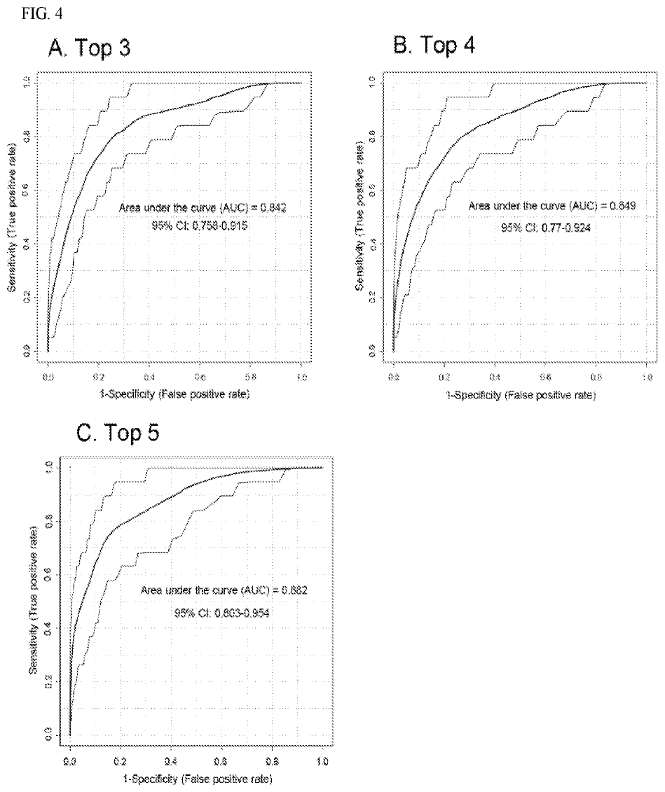

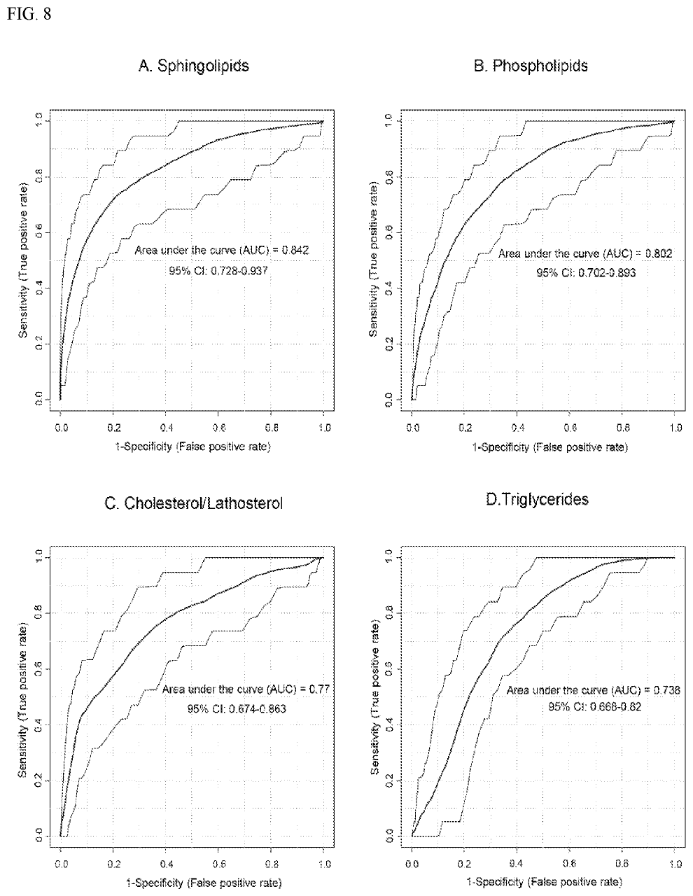

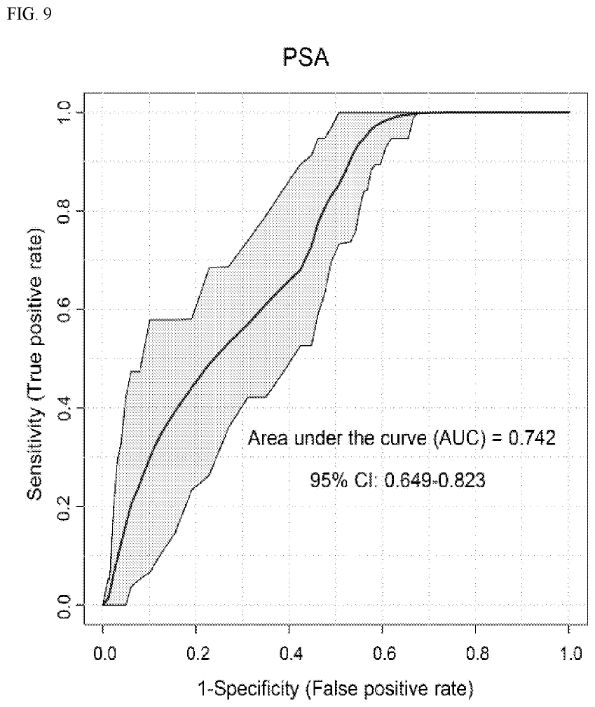

Receiver operator characteristic curves (ROCs) were generated for the four lipid classes identified (sphingolipids [including glycosphingolipids], saturated phospholipids, cholesterol/lathosterol, and triglycerides) to assess the potential of these metabolites to serve as biomarkers for prostate cancer aggressiveness in a binary (low vs intermediate-high) classifier system (FIG. 8A-D). Sphingolipids scored the highest with an area under the curve (AUC) of 0.842 (where 1.000 would indicate no false positives or false negatives). Focusing only on molecular species of sphingolipids, tetrahexosylceramide had AUC 0.815 (0.716-0.896, 95% CI), trihexosylceramide had AUC 0.808 (0.718-0.901, 95% CI), ceramide (d18:1/22:0) had AUC 0.801 (0.722-0.859, 95% CI) and sphingomyelin (d18:0/24:1) had AUC 0.785 (0.694-0.867, 95% CI) (not shown). Combinations of sphingolipids were tested to determine if this improved accuracy. The top three sphingolipids, d18:0/24:1 sphingomyelin, d18:1/16:0 tri and tetrahexosylceramides, yielded AUC 0.842 (CI=0.758-0.915; FIG. 4A). The top four sphingolipids included the addition of d18:1/24:1 ceramide and yielded AUC 0.849 (CI: 0.770-0.924; FIG. 4B). Analysis of the top five most significant sphingolipids yielded AUC 0.882 (CI=0.803-0.954; FIG. 4C) after the addition of d18:1/22:0 ceramide. PSA for the same sample set yielded poorer AUC score of 0.742 (CI=0.649−0.823, FIG. 9) than the 0.882 for the top five most significant sphingolipids. These data indicate that the five-sphingolipid signature are more accurate than PSA in men with existing PCa.

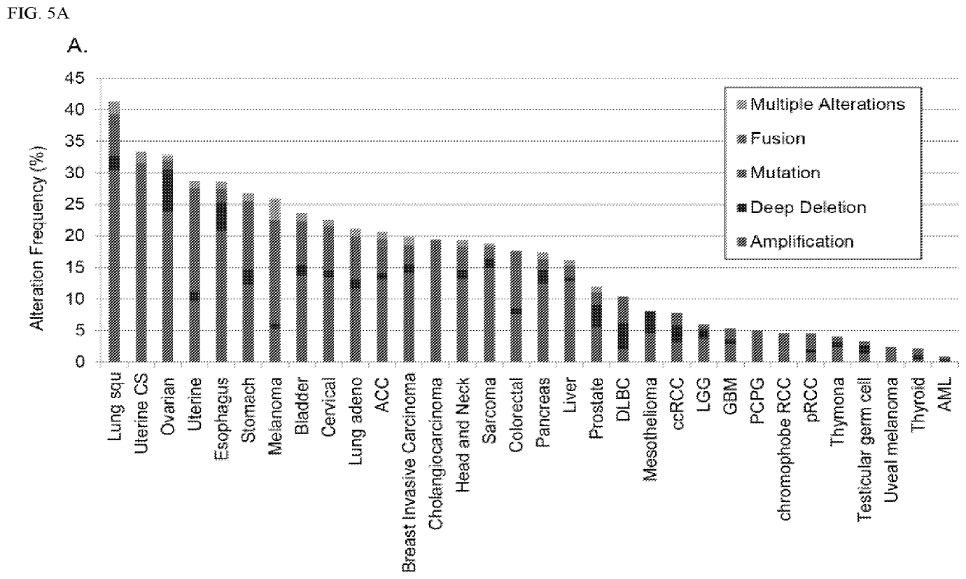

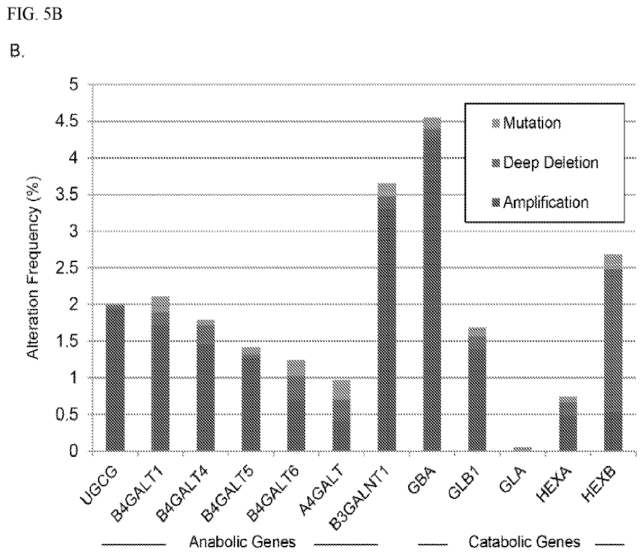

As the two sphingolipid species most associated with aggressiveness were complex glycosphingolipids (tri- and tetrahexosylceramide), the Cancer Genome Atlas (TCGA) PanCancer Atlas data base was examined using cBioPortal to explore whether there were alterations in the genes involved in the metabolism of these molecules from ceramide. Analyses of the anabolic and catabolic genes in this pathway demonstrated alterations (primarily amplifications) for glycosphingolipid genes across several cancer types, which occurred in ˜12% of PCa cancers (FIG. 5A). Lung squamous cell carcinoma had the highest alteration frequency with ˜41% of patients exhibiting alterations in glycosphingolipid pathway genes. High alteration rates (>25%) were found in ovarian, uterine, esophagus, melanoma, and stomach cancers.

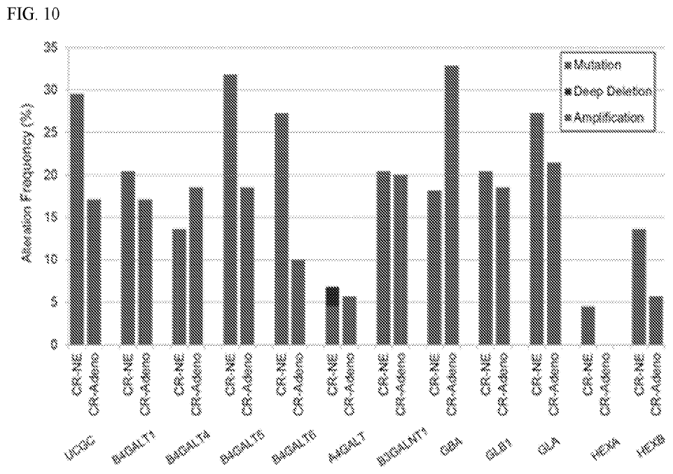

A separate analysis of alterations of individual anabolic (FIG. 5B) and catabolic genes in glycosphingolipid metabolism in all prostate cancer studies available through cBioPortal (Cerami E, Gao J, Dogrusoz U, et al: The cBio cancer genomics portal: an open platform for exploring multidimensional cancer genomics data. Cancer Discov 2:401-4, 2012) demonstrated individual alterations as high as 4.5% throughout the pathway. There was a striking increase in the amplification frequency of B3GALNT1, which is responsible for the biosynthesis of tetrahexosylceramides, and a deep deletion profile for HEXB, the enzyme that removes sugar moieties from tetrahexosylceramides (FIG. 5B). Beltran et al carried out whole exome sequencing of metastatic biopsies (114 metastatic tumor specimens) of castration-resistant prostate cancer or neuroendocrine prostate cancer (Beltran H, Prandi D, Mosquera J M, et al: Divergent clonal evolution of castration-resistant neuroendocrine prostate cancer. Nat Med 22:298-305, 2016). Further analysis of available data from that study revealed amplifications in most glycosphingolipid biosynthetic genes with B4GALT5, which codes for the enzyme that catalyzes the synthesis of lactosylceramide, amplified in ˜30% of those castration resistant biopsies (FIG. 10). The combined data support that biomarker patterns described above may be due, at least in part, to genetic alterations in prostate cancer tissue.

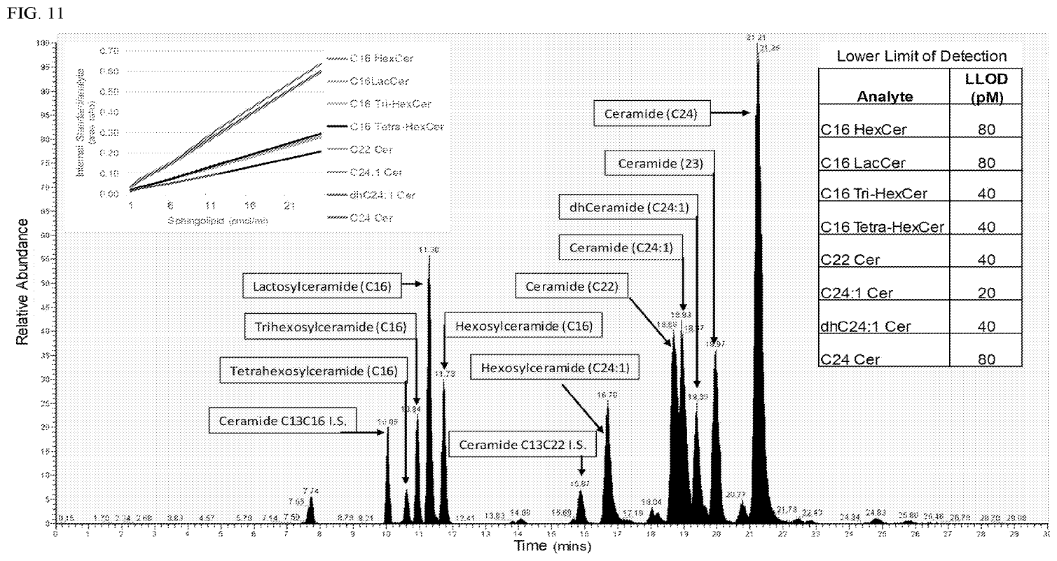

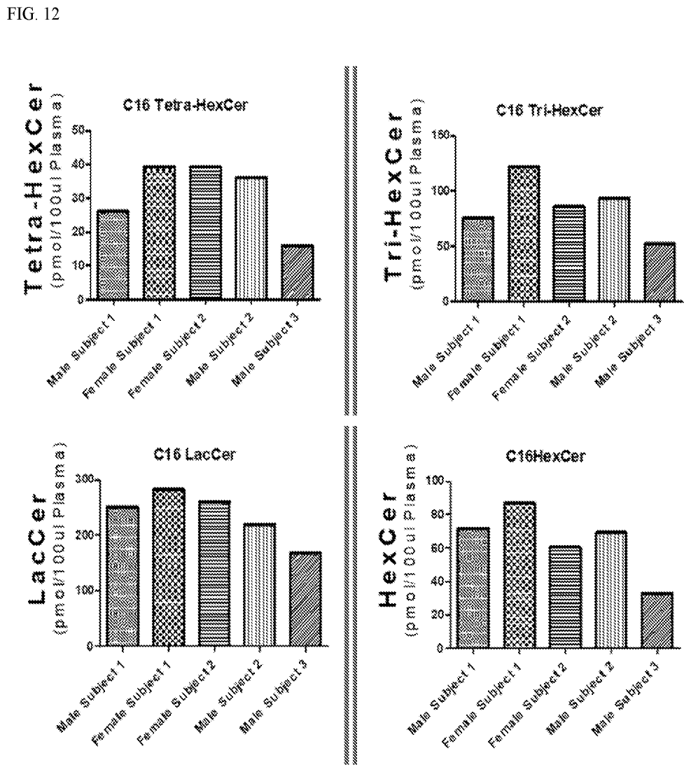

Based on the untargeted metabolomics described above, a targeted mass spectrometry assay was developed to precisely determine the concentrations of the aforementioned metabolites within the glycosphingolipid metabolism pathway. FIG. 11 demonstrates baseline separation and quantification of all major ceramide and glycosylceramide molecular species with lower limits of detection 20-80 μM. FIG. 12 illustrates concentrations (measured by targeted mass spectrometry) of hexosyl-, lactosyl trihexosyl and tetrahexosyl-ceramides in five human subjects. These data demonstrate that targeted metabolomics utilizing mass spectrometry is an extremely sensitive method to measure molecules in biological fluids such as plasma that are associated with cancer severity and aggressiveness.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of measuring metabolites in a subject, comprising:
a) determining the presence, absence, or level of one or more metabolites selected from the group consisting of those described in Table 1 in a sample from a subject.

2. The method of claim 1, wherein said metabolite is a ceramide.

3. The method of claim 2, wherein said ceramide is a tetrahexosylceramide and/or a trihexosylceramide.

4. The method of claim 1, wherein said metabolite is one or more of tetrahexosylceramide, trihexosylceramide, ceramide (d18:1/22:0), SM(d18:0/24:1), cholesterol, cer(d18:1/24:1), C46H80O, TG(14:1/18:1/18:1), lathosterol, C40H75N3O2, PC(20:0/14:0), cer(d18:1/23:0), PC(P-18:1/14:0), PE(P-18:0/22:0), or C16H62N32O29S8.

5. The method of claim 1, wherein said metabolite is one or more of tetrahexosylceramide, (d18:1/16:0), sphingomyelin (d18:1/24:1), trihexosylceramide (d18:1/16:0), ceramide (d18:1/24:1), ceramide (d18:1/22:0), triacylglyceride (14:1/18:1/18:1) lathosterol, heptacosanoic acid, phosphatidylcholine (20:0/14:0), ceramide, phosphatidylethanolamine (d14:2/18:1), sphingomyelin (d18:1/22:0), mulberrofuran E, Ceramide (d18:1/23:0), phosphatidylethanolamine (P-18:0/22:0), cholesterol, glucosylceramide (d18:1/16:0), ceramide (d18:2/23:0), phosphatidylcholine (P-18:1/14:0), triacylglyceride (14:1/14:1/20:1), phosphatidylcholine (18:0/14:0), phosphatidylserine (0-20:0/18:4), glucosylceramide (d18:1/16:0), phosphatidylcholine (A-16:0/16:0), ganglioside GM3 (d18:1/16:0), phosphatidylcholine (P-16:0/16:0), phosphatidylserine (P-20:0/22:4), phosphatidylethanolamine (24:0/P-18:1), ceramide (d18:0/24:1), alpha-Tocopherolquinone, ceramide 1-phosphate (d18:1/26:1), (S)-Abscisic acid, and lactosylceramide (d18:1/16:0).

6. The method of claim 1, wherein said metabolites are tetrahexosylceramide (d18:1/16:0), sphingomyelin (d18:0/24:1), trihexosylceramide (d18:1/16:0), ceramide (d18:1/24:1), and ceramide(d18:1/22:0).

7. The method of claim 1, wherein said metabolites are tetrahexosylceramide (d18:1/16:0), sphingomyelin (d18:0/24:1), trihexosylceramide (d18:1/16:0), and ceramide (d18:1/24:1).

8. The method of claim 1, wherein said metabolites are tetrahexosylceramide (d18:1/16:0), sphingomyelin (d18:0/24:1), and trihexosylceramide (d18:1/16:0).

9. The method of claim 1, wherein said subject has been diagnosed with cancer.

10. The method of claim 1, wherein said subject has not been diagnosed with cancer.

11. The method of claim 1, wherein said subject is undergoing treatment for cancer.

12-13. (canceled)

14. The method of claim 22, wherein said treatment is selected from the group consisting of surgery, radiation, chemotherapy, hormone blocking therapy, and a combination thereof.

15-16. (canceled)

17. The method of claim 1, wherein said determining the presence, absence, or level of said metabolite comprises a chromatography and/or spectrometry method.

18. The method of claim 1, wherein an increased level of said metabolite is indicative of an increased risk of aggressive cancer, an increased likelihood of developing cancer, a poor response to a treatment for cancer, or resistance to a treatment for cancer.

19. (canceled)

20. The method of claim 1, wherein said cancer is selected from the group consisting of prostate cancer, lung squamous cell cancer, uterine cancer, ovarian cancer, esophageal cancer, stomach cancer, and melanoma.

21. (canceled)

22. A method of treating cancer, comprising:
a) determining the presence, absence, or level of one or more metabolites selected from the group consisting of those described in Table 1 in a sample from a subject;
b) providing a recommended treatment based on presence, absence, or level of said metabolites; and
c) administering said treatment to said subject.

23. The method of claim 22, wherein said subject is currently undergoing a treatment for cancer.

24. The method of claim 20, wherein said treatment is continued, discontinued, or altered based on said presence, absence, or level of said metabolites.

25-26. (canceled)

27. The method of claim 22, wherein said cancer is selected from the group consisting of prostate cancer, lung squamous cell cancer, uterine cancer, ovarian cancer, esophageal cancer, stomach cancer, and melanoma.

28-30. (canceled)

31. A method of identifying copy number variation, comprising:
determining the presence of a copy number variation in one or more genes selected from the group consisting of UGCG, B4GALT1, B4GALT5, A4GALT, B3GALTN1, GBA, GLB1, GLA, HEXA, and HEXB in a sample from a subject.

32-34. (canceled)
